SEQUENCE LISTING (9 sequences)

SEQ ID NO: 1 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 2 - protein, 26 aa, Homo sapiens
MDPQTAPSRALLLLLFLHLAFLGGRS

SEQ ID NO: 3 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

SEQ ID NO: 4 - protein, 32 aa, Homo sapiens
SPKMVQGSGCFGRKMDRISSSSGLGCKVLRRH

SEQ ID NO: 5 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 6 - protein, 11 aa, Homo sapiens
  Disulphide bond between Cys resides at positions 5 and 10
ETPDCFWKYCV

SEQ ID NO: 7 - protein, 8 aa, Homo sapiens
  Disulphide bond between Cys resides at positions 2 and 7
ACFWKYCV

SEQ ID NO: 8 - protein, 13 aa, Homo sapiens
LAVMSVDLGSESM

SEQ ID NO: 9 - protein, 13 aa, Homo sapiens
PQTAPSRALLLLL

(19) United States
(12) Patent Application Publication
(10) Pub. No.: US 2006/0183174 A1
(43) Pub. Date: Aug. 17, 2006

(54) Diagnosis

(76) Inventor: Leong Ng, Leicester (GB)

Correspondence Address: STEPTOE & JOHNSON LLP, 1330 CONNECTICUT AVENUE, N.W., WASHINGTON, DC, 20036, US

(21) Appl. No.: 11/055,668

(22) Filed: Feb. 11, 2005

(51) Int. Cl. G01N 33/537

(52) U.S. Cl. 435/7.92

(57) ABSTRACT

A method for screening, diagnosis or prognosis of heart failure in a mammalian subject, for determining the stage or severity of heart failure in a mammalian subject, for identifying a mammalian subject at risk of developing heart failure, or for monitoring the effect of therapy administered to a mammalian subject having heart failure included measuring the level of myeloperoxidase (MPO) in a sample of bodily fluid from the mammalian subject. Methods for monitoring the cardiac health of a mammalian subject are further included. Kits for carrying out such methods are also provided.

The present invention relates to methods for the diagnosis of heart failure in mammalian subjects, such as humans. In particular, it relates to such methods in which the level of myeloperoxidase (MPO) in a sample of bodily fluid is measured.

### BACKGROUND

Heart failure occurs when the heart is not strong enough to pump blood efficiently around the body and becomes increasingly common with age. As a result, fluid collects in the lungs and body tissue and causes congestion (clogging). In many cases, the cause of congestive heart failure is unknown. One common cause can include coronary artery disease. Another common cause can include hypertension. Yet another common cause can include valvular heart disease (especially aortic and mitral disease). Yet other causes can include: infections by viruses (including human immunodeficiency virus), bacteria and parasites; drugs (e.g., doxorubicin [Adriamycin], cyclophosphamide [Cytoxan], cocaine); alcohol; connective tissue disease; infiltrative disease (e.g., amyloidosis, sarcoidosis, hemochromatosis, malignancy); tachycardia; obstructive cardiomyopathy; neuromuscular disease (e.g., muscular or myotonic dystrophy, Friedreich's ataxia); metabolic disorders (e.g., glycogen storage disease type 2 [Pompe's disease] and type 5 [McArdle's disease]); nutritional disorders (e.g., beriberi, kwashiorkor); pheochromocytoma; radiation; endomyocardial fibrosis; eosinophilic endomyocardial disease; high-output heart failure (e.g., intracardiac shunt, atrioventricular fistula, beriberi, pregnancy, Paget's disease, hyperthyroidism, anemia); peripartum cardiomyopathy; and dilated idiopathic cardiomyopathy. The main contributing factor however is age: the heart weakens and blood vessels narrow with age. Therefore, people over the age of 55 are most affected.

Heart failure is often the result of left ventricular systolic dysfunction (LVSD). As many as 40% of patients with clinical heart failure have diastolic dysfunction with normal systolic function. In addition, many patients with systolic dysfunction can have elements of diastolic dysfunction. With systolic dysfunction, the pumping ability of the ventricle is impaired. With diastolic dysfunction, ventricular filling is defective. Ventricular diastolic function depends on the pressure-to-volume relationship in the left ventricle. Decreased compliance of the left ventricular wall leads to a higher pressure for a given diastolic volume. The end result is impaired ventricular filling, inappropriately elevated left atrial and pulmonary venous pressure, and decreased ability to increase stroke volume. These dysfunctions can lead to the clinical syndrome of heart failure.

Heart failure and LVSD are important causes of mortality and morbidity, but diagnosis in the community remains a problem because access to echocardiography is limited by expense, lack of trained personnel and equipment. Screening for LVSD is supported by its prevalence, presence of asymptomatic subjects and availability of therapies. Recent evidence suggests that natriuretic peptides, e.g. B-type natriuretic peptide (BNP) and its precursor N-terminal proBNP(N-BNP), are useful in LVSD screening (McDonagh et al., Lancet 1998; 351:9-13; Hobbs et al., BMJ 2002; 324: 1498-1500; Ng L L et al., Eur J Heart Failure 2003; 5: 775-782) although performance could be sub-optimal in some studies (Vasan et al., JAMA 2002; 288: 1252-1259). However, although sensitive and having high negative predictive values (NPV) for ruling out LVSD, BNP and N-BNP are not especially specific and have low positive predictive values (PPV).

Multimarker approaches especially using inflammatory markers may be useful for boosting specificity of predictions, for example in risk stratification of acute coronary syndromes (Sabatine et al., Circulation. 2002; 105: 1760-3; Brennan et al., N Engl J Med. 2003; 349: 1595-604) where C-reactive protein (CRP) and myeloperoxidase (MPO) have been utilised. CRP predicts future cardiovascular disease (Ridker et al., N Eng J Med 1997;336: 973-979; Ridker et al., N Eng J Med 2000; 342: 836-43). There is also some evidence for elevation of CRP in development of heart failure, with greater values in decompensated cases (Vasan et al., Circulation 2003; 107: 1486-91; Yin et al., Am Heart J 2004; 147(5): 931-8).

Although MPO may have a role in coronary artery disease (Zhang et al., JAMA. 2001; 286: 2136-42) and has been implicated in ventricular remodelling post-infarction (Askari et al., J Exp Med. 2003; 197: 615-24), MPO has not been investigated in human heart failure.

U.S. patent application No. 2002/0164662 discloses that levels of leukocyte and blood MPO are elevated in patients with coronary artery disease. Based on this finding, the authors suggest that MPO can be diagnostic of cardiovascular disease in general, including heart failure. However, no data is presented to support the contention that MPO is indicative of heart failure. In addition, while it is known that MPO is released in coronary artery disease as a result of the recruitment and activation of neutrophils which results in the release of MPO from neutrophils, it is not obvious that MPO would be released in heart failure. In heart failure (stable or with volume overload), there is no data to suggest that measurements of MPO are diagnostic or prognostic. Indeed, the data presented herein proves that the presence of ischaemic heart disease (IHD) is not independently predictive of the presence of elevated MPO levels but that the presence of heart failure does independently predict the presence of raised MPO levels. Patients with heart failure in the examples herein had a variety of underlying causes of heart failure, including hypertension (approximately one third of the heart failure patients). However, MPO was still diagnostic for heart failure independent of whether the patient had current IHD or IHD as the original cause of heart failure. It is demonstrated herein that MPO does not identify patients with IHD from patients without IHD, but does identify patients with heart failure from patients with IHD and patients with heart failure from patients without IHD. Accordingly, it cannot reasonably be said that MPO can be used to diagnose patients with heart failure on the basis that MPO is raised in coronary artery disease. De Pasquale et al, Am J Physiol Heart Circ Physiol 2003, 284: H2136-H2145 discloses that MPO is contained within the lung tissue due to accumulation of neutrophils within the oedematous lung tissue, where ischaemic myocardial damage leads to heart failure. For the reasons discussed above especially the fact that IHD only contributes to the aetiology of heart failure in a proportion of cases, this cannot be extrapolated to its use as a marker of heart failure, whether stable or decompensated.

### SUMMARY

The present invention is based on the surprising and unexpected finding that MPO is diagnostic of heart failure. MPO may be used in combination with a second marker such as CRP, N-BNP, BNP or urotensin to screen for or diagnose heart failure. In addition, it has been found that, surprisingly, CRP and MPO both have incremental value in detection of LVSD together with N-BNP, which can improve the specificity and positive prediction sufficiently to make screening for heart failure a possibility.

In a first aspect, a method for screening, diagnosis or prognosis of heart failure in a mammalian subject, for determining the stage or severity of heart failure in a mammalian subject, for identifying a mammalian subject at risk of developing heart failure, or for monitoring the effect of therapy administered to a mammalian subject having heart failure includes measuring the level of a first marker in a sample of bodily fluid from the mammalian subject, wherein the first marker is myeloperoxidase (MPO). The bodily fluid can be plasma. The level of the first marker is compared with a level of the first marker which is indicative of the absence of heart failure. The level of the first marker which is indicative of the absence of heart failure can be the level of the first marker from one or more mammalian subjects free from heart failure, or a previously determined reference range for the first marker in mammalian subjects free from heart failure. The level of the first marker can be measured by contacting the sample with an antibody that binds specifically to the first marker and measuring any binding that has occurred between the antibody and at least one species in the sample. The antibody may be a monoclonal antibody.

The method may further include measuring the level of a second marker indicative of heart failure. The second marker may be a natriuretic peptide such as brain natriuretic peptide (BNP) or N-terminal pro-brain natriuretic peptide (N-BNP). The second marker may be C-reactive protein (CRP) or urotensin. The level of the second marker may be compared with a level of the second marker which is indicative of the absence of heart failure. For example, a level of the second marker which is indicative of the absence of heart failure can be the level of the second marker from one or more mammalian subjects free from heart failure, or a previously determined reference range for the second marker in mammalian subjects free from heart failure. The level of the second marker may be measured by contacting the sample with an antibody that binds specifically to the second marker and measuring any binding that has occurred between the antibody and at least one species in the sample. The antibody may be a monoclonal antibody.

In another aspect, a method for monitoring the health of a mammalian subject includes measuring the level of a first marker in a sample of bodily fluid from the mammalian subject, wherein the first marker is myeloperoxidase (MPO). In certain embodiments, the level of the first marker is compared with a level of the first marker which is indicative of the absence of heart failure. In other embodiments, the level of the first marker is compared with a level of the first marker which is indicative of worsening of heart failure in a mammalian subject previously determined to be experiencing heart failure. In other embodiments, the level of the first marker is compared with a level of the first marker which is indicative of sudden unexpected cardiac death in a mammalian subject previously determined to be experiencing heart failure. In yet another embodiment, the level of the first marker is compared with a level of the first marker which is indicative of a response to therapy in a mammalian subject previously determined to be experiencing heart failure.

In a further aspect, kits for carrying out such methods are also provided.

Unexpectedly, the level of a first marker, MPO, in a sample of bodily fluid from a mammalian subject is diagnostic of heart failure and can be diagnostic or prognostic of the condition of the subject. In particular, a level of MPO can be used as an initial screen with optional subsequent testing with for a second marker to more efficiently diagnose heart failure, detect a worsening of heart failure, detect deteriorating heart function, or predict the likelihood of sudden unexpected death or response to a therapy in a subject. MPO can be diagnostic for heart failure in a subject without other manifestations of heart disease.

The details of one or more embodiments are set forth in the accompanying drawings and the description below. Other features, objects, and advantages will be apparent from the description and drawings, and from the claims.

### DESCRIPTION OF DRAWINGS

The invention will be described further with reference to the accompanying drawings in which:

FIGS. 1a-c are box plots of plasma MPO, CRP and N-BNP respectively in normal and LVSD subjects;

FIG. 2a is an Receiver Operating Characteristic (ROC) curve for N-BNP, CRP and MPO in LVSD diagnosis. Logistic models 1 (N-BNP, CRP, MPO) and 2 (N-BNP, CRP, MPO, IHD history, gender) are also plotted. FIG. 2b is an ROC curve for N-BNP and CRP in LVSD diagnosis for those with plasma MPO>33.9 ng/ml. A logistic model 1 using N-BNP, CRP, IHD history, gender is also plotted.

FIG. 3 shows the amino acid sequence for human MPO;

FIG. 4 shows the amino acid sequence of proBNP; and

FIG. 5 shows the amino acid sequence for CRP.

### DETAILED DESCRIPTION

MPO has been detected in plasma at higher levels in patients who have heart failure than normal subjects. Measurement of this marker is therefore useful as a diagnostic aid for presence of heart failure and for assessing the severity of heart failure. Measurement of this marker is also useful for monitoring the health of a mammalian subject. MPO, or MPO in combination with a second marker such as CRP, N-BNP, BNP or urotensin, can be used to detect heart failure in patients who are as yet undiagnosed, to detect volume overload (decompensation) in a heart failure patient, to determine whether a patient's condition is worsening (for example changing NYHA Class), to determine whether a heart failure patient's condition is responding to therapy, as a risk factor for developing HF in individuals with Stage A (risk factors relevant to HF) or Stage B (pre-heart failure, e.g. hypertension), and/or as a risk factor for developing sudden cardiac death, e.g. it could be used with other markers and a measure of heart rate variability.

Myeloperoxidase (EC 1.11.1.7) is a major neutrophil protein. It is also present in monocytes. In neutrophils, it is stored in the azurophilic granules and released during phagocytosis. It is a tetrameric, heavily glycosylated heme enzyme of approximately 150 kDa that uses superoxide and hydrogen peroxide generated by oxidative burst in phagocytes to produce hypohalous acid and other reactive oxidants. The human myeloperoxidase nucleotide sequence is 3213 nucleotides in length and contains two different poly-adenylation signals in the 815-nucleotide long 3′ non-coding region. The primary translation product has 745 amino acids, which consists of a pro-sequence (166 amino acids) and a small (122 amino acids) and a large (467 amino acids) subunit of the MPO functional protein. A level of MPO in a sample can be measured by quantifying the amount of MPO in the sample as a whole molecule, as fragments of MPO, or by measuring MPO activity in the sample or a derivative of the sample.

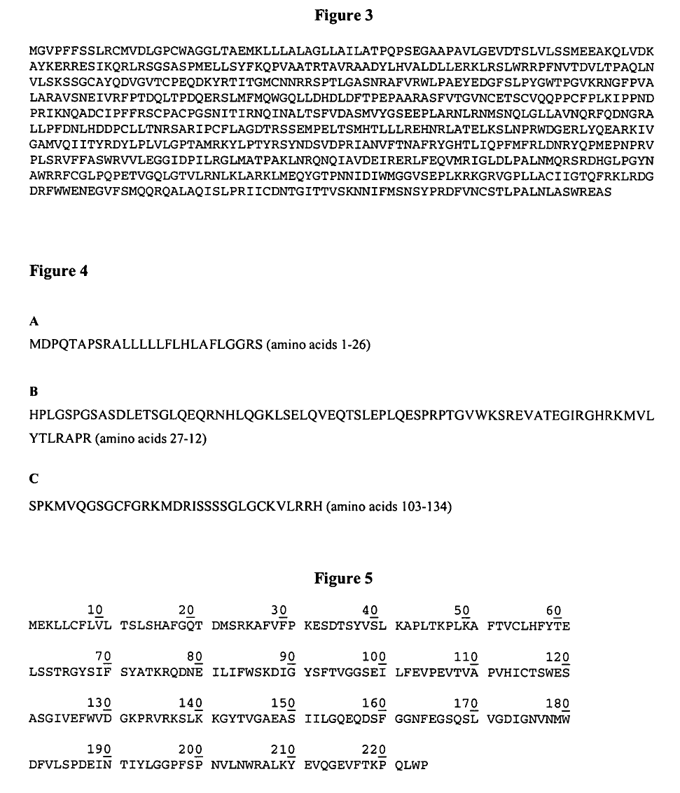

MPO can be identified by its structure, including its polypeptide sequence. For example, the structure can be described by a single polypeptide sequence representing the full polypeptide sequence of MPO, or the structure can be described by a partial sequence, corresponding to a fragment of MPO. A plurality of partial sequences can be combined to make a larger partial sequence or the full sequence. For example, a first peptide sequence can represent the N-terminal polypeptide sequence of MPO, and a second sequence can represent the C-terminal polypeptide sequence of MPO. A fragment of MPO is a fragment of the MPO protein which has an amino acid sequence which is unique to MPO. The fragment may be as few as 6 amino acids, although it may be 7, 8, 9, 10, 11, 12, 13, 14, 15 or more amino acids. The amino acid sequence for human MPO is provided in FIG. 3 (NCBI database Accession NM_{—}000250, Accession AH_{—}002972) and “MPO” as used herein includes variants and allelic variants thereof.

MPO can be modified. The N- or C-terminus can be modified, for example, with a formyl group on the N-terminus. The polypeptide sequence can be modified, (for example, glycosylated, phosphorylated, modified with a hydrophobic group (e.g., myristoylated or geranylgeranylated), or other peptide modification.

In general, an amino acid residue of the peptide can be replaced by another amino acid residue in a conservative substitution. Examples of conservative substitutions include, for example, the substitution of one non-polar (i.e., hydrophobic) residue such as isoleucine, valine, leucine or methionine for another non-polar residue; the substitution of one polar (i.e. hydrophilic) residue for another polar residue, such as a substitution between arginine and lysine, between glutamine and asparagine, or between glycine and serine; the substitution of one basic residue such as lysine, arginine or histidine for another basic residue; or the substitution of one acidic residue, such as aspartic acid or glutamic acid for another acidic residue. In an conservative substitution, an amino acid residue can be replaced with an amino acid residue having a chemically similar side chain. Families of amino acid residues having side chains with chemical similarity have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A conservative substitution may also include the use of a chemically derivatized residue in place of a non-derivatized residue. A chemical derivative a residue chemically derivatized by reaction of a functional group of the residue. Examples of such chemical derivatives include, but are not limited to, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters, or other types of esters or hydrazides. Free hydroxyl groups may be derivatized to form O-acyl or O-alkyl derivatives. Also included as chemical derivatives are those polypeptides which contain one or more naturally-occurring amino acid derivatives of the twenty standard amino acids. For example, 4-hydroxyproline may be substituted for proline; 5-hydroxylsine may be substituted for histidine; homoserine may be substituted for serine; and ornithine may be substituted for lysine.

An amino acid residue of the polypeptide can be replaced by another amino acid residue in a non-conservative substitution. In some cases, a non-conservative substitution will not alter the relevant properties of the polypeptide. The relevant properties can be, without limitation, ability to bind to an antibody that recognizes MPO, or other biological activity.

A level of MPO in a sample can be measured qualitatively or quantitatively using an assay, for example, in an immunochromatographic format. A qualitative assay can be distinguish between the presence or absence of MPO, or can distinguish between categories of MPO levels in a sample, such as absent, low concentration, medium concentration or high concentration, or combinations thereof. A quantitative assay can provide a numerical measure of MPO in a sample. The assay can include contacting MPO with an antibody that recognizes MPO, detecting MPO by mass spectrometry, assaying a sample including cells for expression (e.g., of mRNA or polypeptide) of the MPO gene by the cells, or a combination of measurements. For example, the assay can include contacting a sample with an antibody that recognizes MPO and a mass spectrometry measurement.

In addition or alternatively to measuring the level of MPO protein or fragments thereof, the level of MPO can be measured by measuring the level of MPO-generated oxidative products. Examples of suitable MPO-generated oxidative products are chlorotyrosine, dityrosine, nitrotyrosine and methionine sulphoxide. In addition, MPO-generated lipid peroxidation products may be measured. Suitable MPO-generated lipid peroxidation products are hydroxy-eicosatetraenoic acids (HETEs); hydroxy-octadecadienoic acids (HODEs); F2Isoprostanes; the glutaric and nonanedioic monoesters of 2-lysoPC (G-PC and ND-PC, respectively); the 9-hydroxy-10-dodecenedioic acid and 5-hydroxy-8-oxo-6-octenedioic acid esters of 2-lysoPC (HDdiA-PC and HOdiA-PC, respectively); the 9-hydroxy-12-oxo-10-dodecenoic acid and 5-hydroxy-8-oxo-6-octenoic acid esters of 2-lysoPC(HODA-PC and HOOA-PC, respectively); the 9-keto-12-oxo-10-dodecenoic acid and 5-keto-8-oxo-6-octenoic acid esters of 2-lysoPC (KODA-PC and KOOA-PC, respectively); the 9-keto-10-dodecendioic acid and 5-keto-6-octendioic acid esters of 2-lysoPC (KDdiA-PC and KOdiA-PC, respectively); the 5-oxovaleric acid and 9-oxononanoic acid esters of 2-lysoPC (OV-PC and ON-PC, respectively); 5-cholesten-5α, 6α-epoxy-3β-ol (cholesterol α-epoxide); 5-cholesten-5β, 6α-epoxy-3β-ol (cholesterol β-epoxide); 5-cholesten-3β,7β-diol (7-OH-cholesterol); 5-cholesten-3β, 25-diol (25-OH cholesterol); 5-cholesten-3β-ol-7β-hydroperoxide (7-OOH cholesterol); and cholestan-3β, 5α, 6β-triol (triol).

MPO can be detected in plasma or other bodily fluids which can be obtained from a mammalian body, such as interstitial fluid, urine, whole blood, saliva, serum, lymph, gastric juices, bile, sweat, tear fluid and brain and spinal fluids. Bodily fluids may be processed (e.g. serum) or unprocessed. The mammalian subject may be a human.

The measured level of MPO thereof may be compared with a level of MPO which is indicative of the absence of heart failure. This level may be the level of MPO from one or more mammalian subjects free from heart failure, or with a previously determined reference range for MPO in such mammalian subjects. In this way, the levels can be compared with reference levels determined from population studies of subjects free from heart failure to provide a diagnosis or prognosis. Such subjects may be matched for age and/or gender. In one embodiment, the level of MPO which is indicative of the absence of heart failure may be about 80 pmol/L (or 27 ng/ml) or below, for example, less than 25 ng/ml, less than 23 ng/ml, or less than 20 ng/ml. Levels of MPO which are indicative of heart failure may be about 88 pmol/L (or 30 ng/ml) or more, for example, greater than 32 ng/ml, greater than 35 ng/ml, or greater than 38 ng/ml. One of skill in the art will appreciate that the precise value may vary according to assay format. To monitor the effect of therapy administered to a mammalian subject having heart failure, the measured level of MPO can be compared with a base level for the subject. The base level may be determined prior to commencement of the therapy. Deviations from this base level indicate whether there was an improvement or deterioration of heart failure status and hence whether the therapy is effective. An increased level of MPO indicates worsening heart failure and vice versa. It will be appreciated by one of skill in the art that the respective levels of MPO which are for screening, diagnosis or prognosis of heart failure in a mammalian subject, for determining the stage or severity of heart failure in a mammalian subject, for identifying a mammalian subject at risk of developing heart failure, or for monitoring the effect of therapy administered to a mammalian subject having heart failure may be different from one another. These levels may be easily determined in a manner similar to that described herein.

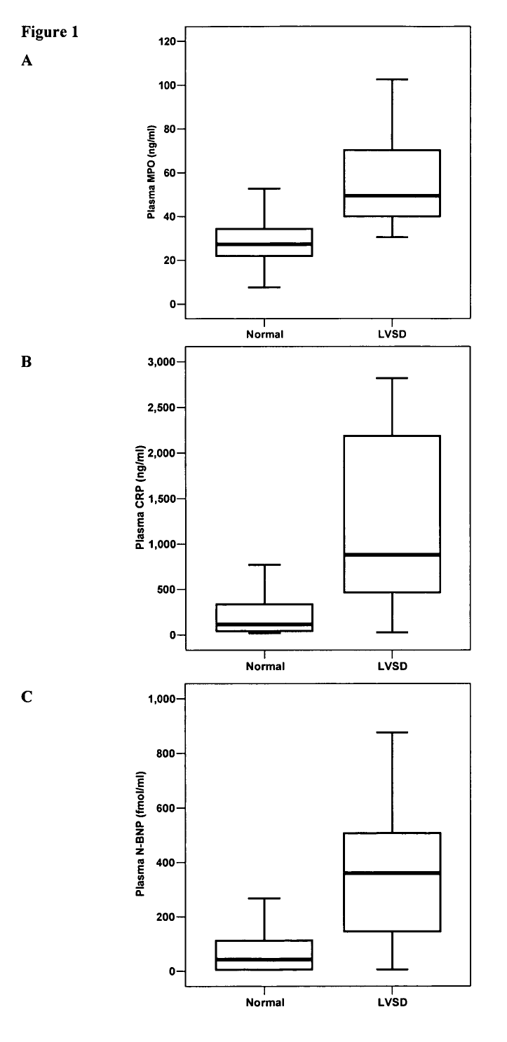

A second marker indicative of heart failure in a mammalian subject may also be measured. The second marker may be C-reactive protein (CRP—SwissProt P02741), Oxygen Regulated Protein (ORP150—NCBI database Accession AAC50947, Accession NP₁₃ 006380), urotensin (UTN—GenBank Accession Numbers NM_{—}021995 and NM_{—}006786), Plasma Surfactant Protein-B (GenBank Accession Number J02761), Nourin-1 (as described in patent application Ser. No. 10/945,442 which is incorporated by reference in its entirety) or a natriuretic peptide, including a native atrial natriuretic peptide (ANP—see Brenner et al, Physiol. Rev., 1990, 70: 665), brain natriuretic peptide (BNP) and C-type natriuretic (CNP—see Stingo et al, Am. J. Physiol. 1992, 263: H1318), and variants or allelic variants thereof. The amino acid sequence of CRP is shown in FIG. 5. A suitable natriuretic peptide is brain natriuretic peptide (BNP) or N-terminal pro-brain natriuretic peptide (N-BNP). In heart failure, there is evidence of upregulation of the Brain natriuretic peptide system, with increased plasma levels of brain natriuretic peptide (BNP) (Wei et al, Circulation 1993; 88: 1004-9; McDonagh et al, Lancet 1998; 351: 9-13), and its precursor N-terminal protein (N-BNP) (Hunt et al, Clin Endocrinol 1997; 47: 287-96; Hughes et al, Clinical Science 1999; 96: 373-380). The whole protein (see FIG. 4) (Sudoh et al, Biochem Biophys Res Commun 1989; 159: 1427-34) consists of a signal peptide sequence (amino acids 1-26), and proBNP (amino acids 27-134), from which is derived the N-BNP (amino acids 27-102) and BNP (amino acids 103-134). The release of proBNP (the intact precursor to the two circulating forms, BNP (the active peptide) and N-BNP (the inactive peptide)) from cardiac myocytes in the left ventricle and increased production of BNP is triggered by myocardial stretch, myocardial tension, and myocardial injury. Another suitable natriuretic peptide is atrial natriuretic peptide (ANP) (Hall, Eur J Heart Fail, 2001, 3:395-397). Urotensin is derived from a prohormone precursor, pro-urotensin (GenBank Accession Number O95399), which is processed to mature urotensin and an N-terminal peptide. The term “urotensin” as used herein further includes pro-urotensin, mature urotensin, its N-terminal derived peptide, its signal peptide, and a C-terminal peptide (Glu-Thr-Pro-Asp-Cys-Phe-Trp-Lys-Tyr-Cys-Val (disulphide bond between Cys⁵ and Cys¹⁰)), as well as fragments thereof. The term urotensin also refers to urotensin related peptide (URP) (Ala-Cys-Phe-Trp-Lys-Tyr-Cys-Val (disulphide bond between Cys² and Cys⁷)) as well as the proform of urotensin related peptide (GenBank Accession Number NM_{—}198152). The use of Plasma Surfactant Protein-B as a marker of heart failure is described in De Pasquale et al, Circulation, 2004, 110:1091-1096.

Fragments of the above-described second markers can be measured. In this case, the fragment will have an amino acid sequence which is unique to the second marker in question. The fragment may be as few as 6 amino acids, although it may be 7, 8, 9, 10, 11, 12, 13, 14, 15 or more amino acids. For ORP150, the fragment may comprise or consist of the sequence LAVMSVDLGSESM. For BNP, the fragment may consist or comprise the sequence PQTAPSRALLLLL.

In a manner similar to the first marker, MPO, the measured level of the second marker may be compared with a level of the second marker which is indicative of the absence of heart failure. This level may be the level of the second marker from one or more mammalian subjects free from heart failure, or with a previously determined reference range for the second marker in mammalian subjects free from heart failure. For example, levels of BNP or N-BNP which are indicative of an increased risk of heart failure may range from 25 fmol/ml or more, for example, greater than 28 fmol/ml, and levels of CRP which are indicative of an increased risk of heart failure may range from 40 ng/ml or more, for example, greater than 42 ng/ml. Again, one of skill in the art will appreciate that the precise values may vary according to assay format.

As is explained in more detail herein, the inventors have demonstrated that using a combination of MPO, CRP and N-BNP provides improved specificity and positive predictive value relative to using any of these markers alone. The result of this is that fewer echocardiographs need to be carried out to confirm heart failure, resulting in a significant cost saving.

Marker levels may be provided in units of concentration, mass, moles, volume or any other measure indicating the amount of marker present.

The respective levels of the first and second markers may be measured using an immunoassay, i.e. by contacting the sample with an antibody that binds specifically to the marker and measuring any binding that has occurred between the antibody and at least one species in the sample. Such assays may be competitive or non-competitive immunoassays. Such assays, both homogeneous and heterogeneous, are well-known in the art, wherein the analyte to be detected is caused to bind with a specific binding partner such as an antibody which has been labelled with a detectable species such as a latex or gold particle, a fluorescent moiety, an enzyme, an electrochemically active species, etc. Alternatively, the analyte may be labelled with any of the above detectable species and competed with limiting amounts of specific antibody. The presence or amount of analyte present is then determined by detection of the presence or concentration of the label. Such assays may be carried out in the conventional way using a laboratory analyser or with point of care or home testing device, such as the lateral flow immunoassay as described in EP291194.

In one embodiment, an immunoassay is performed by contacting a sample from a subject to be tested with an appropriate antibody under conditions such that immunospecific binding can occur if the marker is present, and detecting or measuring the amount of any immunospecific binding by the antibody. The antibody may be contacted with the sample for at least about 10 minutes, 30 minutes, 1 hour, 3 hours, 5 hours, 7 hours, 10 hours, 15 hours, or 1 day. Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, a marker can be detected in a fluid sample by means of a two-step sandwich assay. In the first step, a capture reagent (e.g., an anti-marker antibody) is used to capture the marker. The capture reagent can optionally be immobilised on a solid phase. In the second step, a directly or indirectly labelled detection reagent is used to detect the captured marker. In one embodiment, the detection reagent is an antibody. In another embodiment, the detection reagent is a lectin.

In one embodiment, a lateral flow immunoassay device may be used in the “sandwich” format wherein the presence of sufficient marker in a sample will cause the formation of a “sandwich” interaction at the capture zone in the lateral flow assay. The capture zone as used herein may contain capture reagents such as antibody molecules, antigens, nucleic acids, lectins, and enzymes suitable for capturing MPO and other markers described herein. The device may also incorporate one or more luminescent labels suitable for capture in the capture zone, the extent of capture being determined by the presence of analyte. Suitable labels include fluorescent labels immobilised in polystyrene microspheres. Microspheres may be coated with immunoglobulins to allow capture in the capture zone.

Other assays that may be used include, but are not limited to, flow-through devices.

In a flow-through assay, one reagent (usually an antibody) is immobilised to a defined area on a membrane surface. This membrane is then overlaid on an absorbent layer that acts as a reservoir to pump sample volume through the device. Following immobilisation, the remainder of the protein-binding sites on the membrane are blocked to minimise non-specific interactions. When the assay is used, a bodily fluid sample containing a marker specific to the antibody is added to the membrane and filters through the matrix, allowing the marker to bind to the immobilised antibody. In an optional second step (in embodiments wherein the first reactant is an antibody), a tagged secondary antibody (an enzyme conjugate, an antibody coupled to a coloured latex particle, or an antibody incorporated into a coloured colloid) may be added or released that reacts with captured marker to complete the sandwich. Alternatively, the secondary antibody can be mixed with the sample and added in a single step. If a marker is present, a coloured spot develops on the surface of the membrane.

The term “antibody” as used herein refers to immunoglobulin molecules and immunologically active portions of immunoglobulin molecules, i.e., molecules that contain an antigen binding site that specifically binds an antigen. The immunoglobulin molecules can be of any class (e.g., IgG, IgE, IgM, IgD and IgA) or subclass of immunoglobulin molecule. Antibodies includes, but are not limited to, polyclonal, monoclonal, bispecific, humanised and chimeric antibodies, single chain antibodies, Fab fragments and F(ab′)₂ fragments, fragments produced by a Fab expression library, anti-idiotypic (anti-Id) antibodies, and epitope-binding fragments of any of the above. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. Portions of antibodies include Fv and Fv′ portions.

Antibodies for detecting MPO and the other markers discussed herein are available commercially. For example, anti-MPO and anti-BNP antibodies are available from Abcam Ltd, 21 Cambridge Science Park, Milton Road, Cambridge CB4 0TP, UK, anti-CRP antibodies are available from Alpha Diagnostic International, Inc., 5415 Lost Lane, San Antonio, Tex. 78238 USA and Research Diagnostics Inc Pleasant Hill Road, Flanders N.J. 07836, USA, and anti-ORP150 antibodies are available from Immuno-Biological Laboratories Co. Ltd, 1091-1 Naka, Fujioka-shi, Gunma, 375-0005, Japan. Antibodies binding to BNP and ANP can be obtained commercially. Examples of commercially available antibodies binding to BNP are rabbit anti-human BNP polyclonal antibody (Biodesign International), rabbit anti-BNP amino acids 1-20 polyclonal antibody (Biodesign International), anti-human BNP monoclonal antibody (Immundiagnostik), and rabbit anti-human BNP amino acids 1-10 polyclonal antibody (Immundiagnostik). Examples of commercially available antibodies binding to ANP are mouse anti-human ANP monoclonal antibody (Biodesign International), rabbit anti-human ANP monoclonal antibody (Biodesign International), mouse anti-human ANP monoclonal antibody (Chemicon), rabbit anti-human ANP amino acids 95-103 antibody (Immundiagnostik), rabbit anti-human ANP amino acids 99-126 antibody (Immundiagnostik), sheep anti-human ANP amino acids 99-126 antibody (Immundiagnostik), mouse anti-human ANP amino acids 99-126 monoclonal antibody (Immundiagnostik) and rabbit anti-human a-ANP polyclonal antibody (United States Biological).

The present invention also provides a kit for carrying out the methods of the invention.

Also provided is a kit for screening, diagnosis or prognosis of heart failure in a mammalian subject, for determining the stage or severity of heart failure in a mammalian subject, for identifying a mammalian subject at risk of developing heart failure, or for monitoring the effect of therapy administered to a mammalian subject having heart failure. The kit can include instructions for taking a sample of bodily fluid from the mammalian subject, and one or more reagents for measuring the level of myeloperoxidase (MPO) in the sample.

The one or more reagents may comprise an antibody that binds specifically to the first marker, as is described above. The kit may further comprise one or more reagents for measuring the level of a second marker indicative of heart failure in a mammalian subject, as is also described above.

The instructions for taking a sample of bodily fluid from the mammalian subject may be optional. In addition, a kit may optionally comprise one or more of the following: (1) instructions for using the kit for screening, diagnosis or prognosis of heart failure in a mammalian subject, for determining the stage or severity of heart failure in a mammalian subject, for identifying a mammalian subject at risk of developing heart failure, or for monitoring the effect of therapy administered to a mammalian subject having heart failure; (2) a labelled binding partner to any antibody present in the kit; (3) a solid phase (such as a reagent strip) upon which any such antibody is immobilised; and (4) a label or insert indicating regulatory approval for diagnostic, prognostic or therapeutic use or any combination thereof. If no labelled binding partner to the or each antibody is provided, the or each antibody itself can be labelled with a detectable marker, e.g., a chemiluminescent, enzymatic, fluorescent, or radioactive moiety.

In a further aspect, myeloperoxidase (MPO) is used as a marker of heart failure in a mammalian subject. A reagent for measuring the level of myeloperoxidase (MPO) may be used in a sample in the manufacture of a diagnostic for screening, diagnosis or prognosis of heart failure in a mammalian subject, for determining the stage or severity of heart failure in a mammalian subject, for identifying a mammalian subject at risk of developing heart failure, or for monitoring the effect of therapy administered to a mammalian subject having heart failure.

Preferred features of each aspect of the invention are as for each of the other aspects mutatis mutandis. The prior art documents mentioned herein are incorporated to the fullest extent permitted by law.

The invention will be described further in the following, non-limiting example.

Methods

Patient Recruitment

Randomly selected men (45-80 years) and women (55-80 years) from 21 general practices in Leicestershire (population approximately 1 million) were invited for screening (between September, 1999 and May, 2002). All subjects with a prior history of heart failure or LVSD were excluded. Subjects gave informed consent and the study was approved by the Leicestershire Research Ethics Committee.

Echocardiography

Patients attended the Leicester Royal Infirmary for echocardiography and blood sampling. Transthoracic echocardiography was performed by one operator using a Sonos 5500 instrument (Philips Medical Systems). Full details of the scanning protocol have been described (Ng L L et al., Eur J Heart Failure 2003; 5: 775-782). A 16 segment wall motion index (LVWMI) based on the American Society of Echocardiography model (Schiller et al., J Am Soc Echocardiogr 1989; 2: 358-67) was calculated by 3 investigators blinded to the peptide measurements. Those with a LVWMI score of ≧1.8 (equivalent to a LV ejection fraction (LVEF) of 40%) (Ng L L et al., Eur J Heart Failure 2003; 5: 775-782) were considered to have LVSD. ECGs with the following major abnormalities were noted:—atrial fibrillation, LV hypertrophy, Left bundle branch block, Q wave.

Laboratory Methods

20 ml of venous blood was collected into pre-chilled Na-EDTA tubes containing aprotinin and plasma stored at −70° C. until assay. Plasma was assayed for N-BNP, CRP and MPO using non-competitive immunoluminometric assays. The N-BNP assay has been previously described (Ng L L et al., Eur J Heart Failure 2003; 5: 775-782; Omland et al., Circulation 2002; 106: 2913-8). CRP was measured using an ELISA plate immobilised monoclonal CRP antibody (100 ng/l 00L) (Unipath PLC, Bedford) and a rabbit polyclonal antibody (50 ng/100 μL) (Merck BioSciences, Nottingham, UK). The MPO assay employed an ELISA plate immobilised monoclonal MPO antibody (100 ng/100 μL) (Research Diagnostics Inc., Flanders, N.J., USA) and a rabbit polyclonal antibody (50 ng/100 μL) (Merck BioSciences, Nottingham, UK). Detection employed a secondary reagent (biotinylated anti-rabbit IgG from Sigma, Poole, UK, diluted 1:250,000) and methyl-acridinium ester labeled streptavidin (Omland et al., Circulation 2002; 106: 2913-8). All immunoluminometric assays were read on a Dynex MLX luminometer, with sequential injections of 100 mmol/l HNO₃ containing 0.05% hydrogen peroxide, and 250 mmol/l NaOH. The CRP and MPO assays had inter- and intra-assay coefficients of variation under 10%, with lower limits of detection of 20 and 0.25 ng/ml respectively.

Statistical Analysis

Statistical analysis was performed using SPSS version 12.0 (SPSS Inc, IL). Medians [ranges] are reported and comparisons were performed using the Mann-Whitney or χ² test. Spearman correlation coefficients (rₛ) and the area under the receiver-operator-characteristic (ROC) curves (AUC) and their associated 95% confidence intervals were estimated. General linear model and binary logistic regression analysis were performed on SPSS with the stated variables, with a constant included in the models. In regression, probability for entry or removal was set at P<0.05 and P<0.10 respectively (when using forward or backward likelihood ratio regression analysis).

Results

Of the 2392 subjects approached, screening was accepted by 1360. The number of participants with an analysable echocardiography scan and blood samples was 1331 (Table 1). The 28 subjects (2.1% of the whole population) with previously undiagnosed LVSD as defined above (LVWMI≧1.8 or LVEF≦40%) had elevated plasma N-BNP compared to normal subjects (Table 1, FIG. 1). Plasma CRP and MPO levels were also very significantly elevated in the LVSD (Table 1, FIG. 1). Correlations between plasma N-BNP and CRP (rₛ=0.071, P<0.0005) and MPO (rₛ=0.139, P<0.0005) were modest, as was that between plasma CRP and MPO (rₛ=0.168, P<0.0005). LVWMI score was correlated to plasma N-BNP, CRP and MPO (rₛ=0.145, 0.168, 0.113 respectively, P<0.0005 for all). Plasma CRP was elevated in smokers (median[range] 161.5[20-3935.8] ng/ml compared to non-smokers 88.8[20-3012.2] ng/ml), but plasma MPO was not affected by smoking.

Plasma N-BNP, MPO and CRP were log-transformed for further analyses. In univariate analysis, plasma MPO was also elevated in those with an ischaemic heart disease (IHD) history, those with major ECG abnormalities and older subjects (Table 2). Similarly, plasma CRP was elevated in these groups and also in hypertensives, females and those with a ponderal index above a median of 26.22 kg/m² (Table 2). However, in multivariate analysis using the general linear model (SPSS), the only factor that independently affected both CRP and MPO levels was presence of LVSD (Table 2). Of particular note was the lack of influence of IHD history independent of that of presence of LVSD (heart failure) on MPO levels.

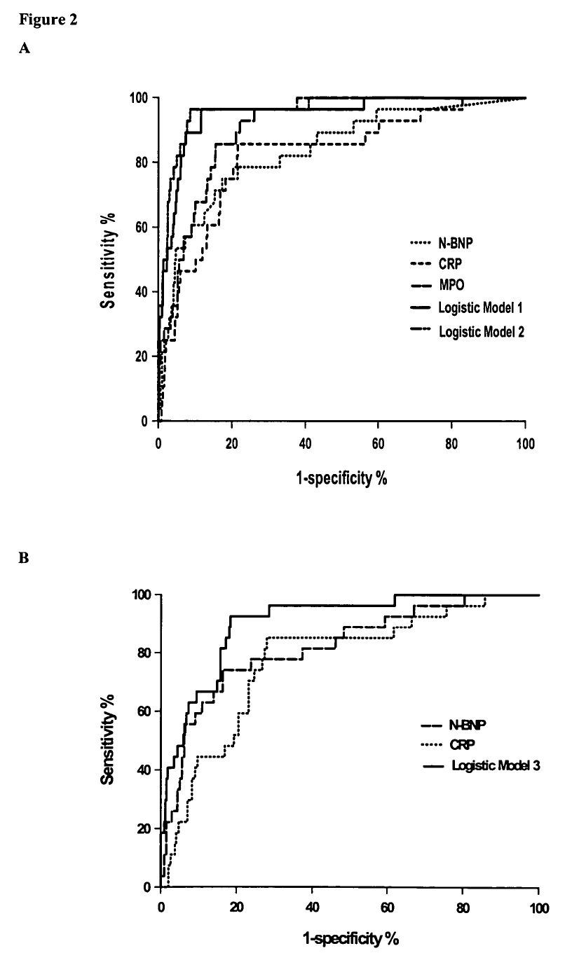

For the diagnosis of LVSD, the area under the Receiver Operating Characteristic curves (ROC AUCs) were significantly different from the diagonal (P<0.0005) for all 3 markers (FIG. 2, Table 3). Individual markers had high sensitivity and NPV, but lower specificites and PPV (with MPO performing better than either CRP or N-BNP). The number of subjects that had to be examined by echocardiography to diagnose 1 LVSD case was 13.5 for MPO compared to 29.7 (N-BNP) and 35.5 (CRP).

Using binary logistic regression analysis for LVSD diagnosis using all 3 peptides, a model accounting for a Nagelkerke r² of 0.448 (P<0.0005) was constructed with all 3 peptides contributing independently (Table 4, logistic model 1). The predicted probability (or C-statistic) when plotted as a ROC curve yielded an area of 0.949, with improved specificity and PPV (Table 3). The number of subjects needed to scan was halved compared to using MPO alone.

All 3 peptides and clinical characteristics (age, gender, history of IHD, hypertension or diabetes) were entered into binary logistic regression analysis to examine whether these readily-available factors and variables associated with LVSD could further improve the diagnostic algorithm. In addition to the 3 peptides, the only significant independent predictors were male gender and IHD history (Table 4, logistic model 2) accounting for a Nagelkerke r² of 0.52 (P<0.0005). The improved specificity and PPV (Table 3) would justify these simple inclusions. Only about 4 subjects needed to be scanned to detect 1 case of LVSD.

Plasma MPO had the best values for specificity and PPV when considered alone. At a cut-off level of plasma MPO level at 33.9 ng/ml, 27 out of 28 LVSD patients would be recovered with 338 other normal subjects (n=365). The utility of CRP, N-BNP and logistic models with these 2 peptides in those with MPO levels above 33.9 ng/ml was examined. FIG. 2 illustrates ROC curves for CRP and N-BNP, and Table 5 documents the ROC AUCs, specificities, PPV of these peptides, and number of cases needing echocardiography. A logistic model with N-BNP, CRP, IHD history accounted for a Nagelkerke r² of 0.43 (P<0.0005), with odds ratios of 10.06 [P<0.0005], 5.41 [P<0.0005], 3.93 [P<0.01] and 4.17 [P<0.007] respectively. Although both N-BNP and CRP individually reduced the number of scans needed, the logistic model (Ng L L et al., Eur J Heart Failure 2003; 5: 775-782) with N-BNP, CRP, IHD history and gender achieved the largest reduction (Table 5).

The costings for a screening programme involving plasma N-BNP, MPO and CRP alone and in combination using logistic models 1 and 2 above are illustrated in Table 6. Costings are based on a recent cost-effectiveness analysis by Heidenreich et al (Heidenreich et al., J Am Coll Cardiol 2004; 43: 1019-26), assuming echocardiography scans and plasma N-BNP cost US$420 and 32 respectively. We have assumed MPO and CRP tests costs were similar to N-BNP. The findings suggest logistic model 2 (strategy F) may detect LVSD at lowest cost per case detected. If plasma MPO (>33.9 ng/ml) was used as an initial screen, subsequent testing with N-BNP or CRP achieved some cost reduction for LVSD detection, but the largest reduction was with both these markers in a logistic model (Ng L L et al., Eur J Heart Failure 2003; 5: 775-782) with gender and IHD history.

Discussion

Interest in screening for LVSD has been facilitated by the introduction of tests for B-type natriuretic peptides (McDonagh et al., Lancet 1998; 351:9-13; Hobbs et al., BMJ 2002; 324: 1498-1500; Ng L L et al., Eur J Heart Failure 2003; 5: 775-782; Vasan et al., JAMA 2002; 288: 1252-1259), although its efficacy has been questioned due to a relatively low specificity in some studies (Vasan et al., JAMA 2002; 288: 1252-1259). In a cost-effectiveness analysis, Heidenreich et al (Heidenreich et al., J Am Coll Cardiol 2004; 43: 1019-26) suggest that, at a population prevalence of 1% LVSD, it is cost-effective to screen with B-type natriuretic peptides even with its limited specificity. The present study, which excluded all known patients with LVSD, detected a population prevalence of 2.1%, a level that may justify screening. Any screening strategy that could improve the specificity of N-BNP may reduce costs further.

Using markers that are associated with cardiovascular disease (MPO and CRP), the increased specificity of MPO compared to N-BNP in LVSD screening was demonstrated. CRP did not perform as well as N-BNP. The only independent predictor of plasma MPO and CRP in this predominantly healthy population was presence of LVSD. Logistic models confirmed the independent diagnostic role of all 3 markers, which could be improved by factors such as presence of IHD and male gender. Using this final logistic model could achieve significant savings (down to a third of performing echocardiography scans on the whole population, or half that of plasma N-BNP alone as an initial screening test with echocardiography of those with positive tests). However, the most significant savings are accrued from use of plasma MPO as an initial screening test, followed by N-BNP and CRP in a logistic model with these markers, IHD history and gender. The latter two factors are readily available and improve specificity of LVSD diagnosis.

Conclusions

In this community cohort, analysis of inflammatory markers such as CRP and MPO improved the specificity of plasma N-BNP in detection of previously undiagnosed LVSD. Costs of a screening programme can be minimized by using plasma MPO as an initial screening test, and then CRP and N-BNP in a logistic model with gender and IHD history. Such a strategy reduces the cost of a proven cost-effective screening strategy using plasma N-BNP by 2-3 fold.

TABLE 1
Study Population Characteristics (n = 1331)
Values are numbers (%) unless specified otherwise.

| | ALL | No LVSD | LVSD |
|---|---|---|---|
| Men/Women: | 752:579 | 730:573 | 22:6 |
| | (56.5:43.5) | (56.0:42.0) | (78.6:21.4) * |
| Age (in years): | 63 (45 | 63 (45 | 68 (51 |
| mean (range) | to 80) | to 80) | to 80) § |
| Practice Jarman | +7.05 | +7.05 | +8.51 |
| score: mean (range) | (−16.0 | (−16.0 | (−10.9 |
| | to +41.4) | to +41.4) | to +41.4) |
| Body Mass Index | 26.8 ± 4.5 | 26.7 ± 4.4 | 27.2 ± 5.3 |
| (kg/m²) | | | |
| Systolic blood | 135 ± 19 | 135 ± 19 | 138 ± 19 |
| pressure: | | | |
| mean ± SD | | | |
| Diastolic blood | 78 ± 12 | 78 ± 12 | 79 ± 14 |
| pressure: | | | |
| mean ± SD | | | |
| Current smoker | 261 (19.6) | 254 (19.5) | 7 (25) |
| Plasma Creatinine | 89.5 ± 30.1 | 89.3 ± 30.3 | 99.9 ± 18.3 ∥ |
| Medical History | | | |
| Myocardial infarction | 33 (2.5) | 27 (2.0) | 6 (21.4) |
| Angina | 92 (6.9) | 84 (6.3) | 8 (28.6) † |
| Hypertension | 325 (24.4) | 317 (23.8) | 8 (28.6) |
| Diabetes mellitus | 64 (4.8) | 62 (4.7) | 2 (7.1) |
| Other Cardiac | | | |
| Abnormalities | | | |
| AF | 18 | 16 | 2 |
| ECG LVH | 124 | 121 | 3 |
| Valvular | 9 | 8 | 1 |
| abnormalities ‡ | | | |
| Prescribed therapy | | | |
| ACE inhibitor/ARB | 118 (8.9) | 112 (8.6) | 6 (21.4) * |
| Loop diuretic | 36 (2.7) | 34 (2.6) | 2 (7.1) |
| Other diuretic | 165 (12.4) | 163 (12.5) | 2 (7.1) |
| Beta-blocker | 152 (11.4) | 148 (11.4) | 4 (14.3) |
| Nitrate | 53 (4.0) | 46 (3.5) | 7 (25) † |
| Calcium channel | 134 (10.1) | 128 (9.8) | 6 (21.4) * |
| blocker | | | |

Natriuretic peptides and inflammatory markers (median (range)

| | | | |
|---|---|---|---|
| Plasma N-BNP | 44.9 (5.7 | 42.5 (5.7 | 360.3 (5.7 |
| (fmol/ml) | to 1230.3) | to 1166.4) | to 1230.2) ∥ |
| Plasma MPO (ng/ml) | 27.6 (7.7 | 27.3 (7.7 | 49.5 (30.5 |
| | to 156.9) | to 156.9) | to 102.5) ∥ |
| Plasma CRP (ng/ml) | 121.5 (20 | 116.1 (20 | 880.5 (27.4 |
| | to 4469.8) | to 4469.8) | to 2817.6) ∥ |

‡ Valvular abnormalities include moderate/severe mitral regurgitation or aortic stenosis
P values for comparisons between LVSD and no LVSD groups:
Mann Whitney test
∥ P < 0.0005
§ P < 0.005
χ² test
† P < 0.001
* P < 0.05

TABLE 2
Univariate and multivariate determinants of plasma MPO and CRP. Median (ranges) are reported.

| | Protein level (ng/ml) Univariate Analysis | | | General Linear Model |
|---|---|---|---|---|
| | | | Univariate | Multivariate |
| | | | analysis | analysis |
| | Without factor tested | With factor tested | P value* | P value† |
| Plasma MPO (ng/ml) | | | | |
| Left Ventricular Systolic Dysfunction | 27.3 (7.7-156.9) | 49.5 (30.5-102.5) | 0.0005 | 0.0005 |
| Ischaemic Heart Disease | 27.4 (7.7-112.6) | 30.5 (12.3-156.9) | 0.001 | NS |
| Major ECG abnormalities | 27.2 (7.7-156.9) | 30.0 (10.2-92.1) | 0.001 | NS |
| Age < median (62.9 yrs) | 26.8 (7.7-156.9) | 49.5 (30.5-102.5) | 0.0005 | NS |
| Hypertension | 27.4 (7.7-102.5) | 28.5 (8.2-156.9) | NS | NS |
| Diabetes | 27.6 (9.7-156.9) | 27.2 (13.4-69.0) | NS | NS |
| Male Gender | 28.0 (8.2-156.9) | 27.4 (7.7-102.5) | NS | NS |
| Ponderal index<median (26.2 kg/m²) | 27.1 (7.7-156.9) | 28.2 (8.0-112.6) | NS | NS |
| Plasma CRP (ng/ml) | | | | |
| Left Ventricular Systolic Dysfunction | 116.1 (20.0-4469.8) | 880.5 (27.4-2817.6) | 0.0005 | 0.0005 |
| Ischaemic Heart Disease | 116.1 (20.0-4469.8) | 176.2 (20.0-2670.4) | 0.011 | NS |
| Major ECG abnormalities | 113.3 (20.0-4469.8) | 161.5 (20.0-2983.9) | 0.002 | NS |
| Age < median (62.9 yrs) | 106.2 (20.0-3012.2) | 136.4 (20.0-4469.8) | 0.006 | NS |
| Hypertension | 109.5 (20.0-4469.8) | 161.4 (20.0-3232.4) | 0.0005 | NS |
| Diabetes | 119.8 (20.0-4469.8) | 140.9 (20.0-2983.9) | NS | NS |
| Male Gender | 133.0 (20.0-3935.8) | 104.4 (20.0-4469.8) | 0.03 | NS |
| Ponderal index<median (26.2 kg/m²) | 88.4 (20.0-3232.4) | 148.3 (20.0-4469.8) | 0.0005 | NS |

*Univariate P value (Mann-Whitney test)
†Multivariate P value (from General linear model)

TABLE 3
ROC-AUCs for plasma N-BNP, MPO and CRP for diagnosis of LVSD with sensitivities, specificities and positive and negative
predictive values (PPV and NPV). Total number of echocardiography scans needed (% of the initial population) and number of
subjects that have to be scanned in order to detect a case of LVSD are also reported. The ROC-AUCs for logistic models using
plasma proteins with or without clinical details are reported below. A sensitivity of 96.4% will miss 1 LVSD case.

| | | 95% | | | | | | Number of | Number to Echo |
|---|---|---|---|---|---|---|---|---|---|
| | ROC | Confidence | Cut-off | % | % | | | Echos needed | to detect 1 |
| Peptide(s) | AUC | Interval | value | sensitivity | specificity | % PPV | % NPV | (%) | case of LVSD |
| Plasma N-BNP | 0.839 | 0.759-0.920 | 26.2 | 96.4 | 40.5 | 3.4 | 99.8 | 802 (60.3%) | 29.7 |
| (fmol/ml) | | | | | | | | | |
| Plasma MPO | 0.909 | 0.874-0.919 | 33.9 | 96.4 | 74.1 | 7.4 | 99.9 | 365 (27.4%) | 13.5 |
| (ng/ml) | | | | | | | | | |
| Plasma CRP | 0.824 | 0.742-0.906 | 47.3 | 96.4 | 28.5 | 2.8 | 99.7 | 958 (72.0%) | 35.5 |
| (ng/ml) | | | | | | | | | |
| Logistic model 1 | 0.949 | 0.910-0.988 | 0.024* | 96.4 | 88.4 | 15.2 | 99.9 | 178 (13.4%) | 6.6 |
| (N-BNP, MPO, CRP) | | | | | | | | | |
| Logistic model 2 | 0.962 | 0.934-0.991 | 0.032* | 96.4 | 91.3 | 19.2 | 99.9 | 140 (10.5%) | 5.2 |
| (N-BNP, MPO, CRP, | | | | | | | | | |
| IHD history, gender) | | | | | | | | | |

*Predicted probability from logistic model

TABLE 4
Binary logistic regression analysis of various predictors of LVSD.
P values and Odds ratios (for ten-fold change in peptide level)
are reported for the predictor variables and factors. B and SEM
refer to the regression coefficient and its standard error.

| | | | |
|---|---|---|---|
| B | SEM | P value | Odds Ratio |

Model 1

| | | | | |
|---|---|---|---|---|
| Log₁₀ N-BNP | 2.070 | 0.509 | 0.0005 | 7.922 |
| Log₁₀ MPO | 6.526 | 1.300 | 0.0005 | 682.735 |
| Log₁₀ CRP | 1.524 | 0.398 | 0.0005 | 4.589 |

Model 2

| | | | | |
|---|---|---|---|---|
| Log₁₀ N-BNP | 1.964 | 0.529 | 0.0005 | 7.129 |
| Log₁₀ MPO | 7.171 | 1.436 | 0.0005 | 1301.7 |
| Log₁₀ CRP | 1.668 | 0.428 | 0.0005 | 5.303 |
| Male gender | 1.288 | 0.544 | 0.02 | 3.625 |
| Ischaemic Heart | 1.386 | 0.549 | 0.012 | 3.999 |
| Disease | | | | |

| | |
|---|---|
| Age, Hypertension, Diabetes | NS |

TABLE 5
ROC-AUCs for plasma N-BNP and CRP in subjects with plasma MPO > 33.9 ng/ml (n = 365), for diagnosis of LVSD with sensitivities,
specificities. positive and negative predictive values (PPV and NPV). Total number of echocardiography scans needed (% of the
initial population) and number of subjects that have to be scanned in order to detect a case of heart failure are also reported.
The ROC-AUCs for a logistic model (3) using plasma CRP, N-BNP, IHD history and gender are reported below.

| | | 95% | | | | | | Number of | Number to Echo |
|---|---|---|---|---|---|---|---|---|---|
| | ROC | Confidence | Cut-off | % | % | | | Echos needed | to detect 1 |
| Peptide(s) | AUC | Interval | value | sensitivity | specificity | % PPV | % NPV | (%) | case of LVSD |
| Plasma N-BNP | 0.821 | 0.734-0.908 | 5.8 | 100 | 19.6 | 9.0 | 100 | 299 (22.4%) | 11.1 |
| (fmol/ml) | | | | | | | | | |
| Plasma CRP | 0.773 | 0.684-0.863 | 27.3 | 100 | 14.2 | 8.5 | 100 | 317 (23.8%) | 11.7 |
| (ng/ml) | | | | | | | | | |
| Logistic model 3 | 0.904 | 0.853-0.956 | 0.0052* | 100 | 40.0 | 11.4 | 100 | 267 (17.8%) | 8.7 |
| (N-BNP, MPO, CRP, | | | | | | | | | |
| IHD history, gender) | | | | | | | | | |

*Predicted probability from logistic model

TABLE 6
A number of different strategies for screening are
analysed for the total number and costs of scans and
N-BNP, CRP or MPO tests utilised for screening.

| | | | | | | | Cost to |
|---|---|---|---|---|---|---|---|
| | | Total | Cost of | Cost of | | | detect 1 |
| Strat- | Plasma | Echo | Echo | plasma | Total | Cost/1000 | case |
| egy | tests | scans | scans $ | tests $ | Cost $ | subjects | LVSD $ |
| A | | 1331 | 559020 | 0 | 559020 | 420000 | 15000 |
| B | 1331 | 802 | 336960 | 42592 | 379552 | 285163 | 10562 |
| C | 1331 | 958 | 402631 | 42592 | 445223 | 334503 | 12389 |
| D | 1331 | 365 | 153299 | 42592 | 195891 | 147176 | 5451 |
| E | 3993 | 178 | 74822 | 127776 | 202598 | 152215 | 5638 |
| F | 3993 | 140 | 58951 | 127776 | 186727 | 140291 | 5196 |
| G | 1696 | 299 | 125498 | 54272 | 179770 | 135064 | 4823 |
| H | 1696 | 317 | 133080 | 54272 | 187352 | 140761 | 5027 |
| I | 2061 | 237 | 99380 | 65952 | 165332 | 124217 | 4436 |

Strategies
A: Scan all patients
B: Plasma N-BNP tests in all patients, scan the positive tests
C: Plasma CRP tests in all patients, scan the positive tests.
D: Plasma MPO tests in all patients, scan the positive tests.
E: Logistic model 1 with N-BNP, CRP, MPO. Scan the positive tests
F: Logistic model 2 with N-BNP, CRP, MPO, gender, IHD history. Scan the positive tests.
G: Plasma MPO tests in all patients, plasma N-BNP in MPO positive cases, scan the positive tests from sequential plasma testing.
H: Plasma MPO tests in all patients, plasma CRP in MPO positive cases, scan the positive tests from sequential plasma testing.
E: Plasma MPO tests in all patients, plasma CRP and N-BNP in MPO positive cases. Use logistic model 3 with CRP, N-BNP, gender, IHD history to detect positives for scanning

1. A method for screening, diagnosis or prognosis of heart failure in a mammalian subject, for determining the stage or severity of heart failure in a mammalian subject, for identifying a mammalian subject at risk of developing heart failure, or for monitoring the effect of therapy administered to a mammalian subject having heart failure, said method comprising:
measuring the level of a first marker in a sample of bodily fluid from said mammalian subject, wherein said first marker is myeloperoxidase (MPO).

2. The method of claim 1, wherein the bodily fluid is plasma.

3. The method of claim 1, wherein the level of the first marker is compared with a level of the first marker which is indicative of the absence of heart failure.

4. The method of claim 3, wherein the level of the first marker which is indicative of the absence of heart failure is the level of the first marker from one or more mammalian subjects free from heart failure, or a previously determined reference range for the first marker in mammalian subjects free from heart failure.

5. The method of claim 1, wherein the level of the first marker is measured by contacting the sample with an antibody that binds specifically to the first marker and measuring any binding that has occurred between the antibody and at least one species in the sample.

6. The method of claim 5, wherein the antibody is a monoclonal antibody.

7. The method of claim 1, further comprising measuring the level of a second marker indicative of heart failure.

8. The method of claim 7, wherein the second marker is a natriuretic peptide.

9. The method of claim 8, wherein the natriuretic peptide is brain natriuretic peptide (BNP) or N-terminal pro-brain natriuretic peptide (N-BNP).

10. The method of claim 7, wherein the second marker is C-reactive protein (CRP).

11. The method of claim 7, wherein the second marker is urotensin.

12. The method of claim 7, wherein the level of the second marker is compared with a level of the second marker which is indicative of the absence of heart failure.

13. The method of claim 12, wherein the level of the second marker which is indicative of the absence of heart failure is the level of the second marker from one or more mammalian subjects free from heart failure, or a previously determined reference range for the second marker in mammalian subjects free from heart failure.

14. The method of claim 7, wherein the level of the second marker is measured by contacting the sample with an antibody that binds specifically to the second marker and measuring any binding that has occurred between the antibody and at least one species in the sample.

15. The method of claim 14, wherein the antibody is a monoclonal antibody.

16. A kit for carrying out the method of claim 1.

17. A kit for screening, diagnosis or prognosis of heart failure in a mammalian subject, for determining the stage or severity of heart failure in a mammalian subject, for identifying a mammalian subject at risk of developing heart failure, or for monitoring the effect of therapy administered to a mammalian subject having heart failure, said kit comprising:
instructions for taking a sample of bodily fluid from said mammalian subject; and
one or more reagents for measuring the level of myeloperoxidase (MPO) in the sample.

18. A kit of claim 17, wherein the bodily fluid is plasma.

19. A kit of claim 17, wherein the one or more reagents comprise an antibody that binds specifically to the first marker.

20. A kit of claim 19, wherein the antibody is a monoclonal antibody.

21. A kit of claim 17, further comprising one or more reagents for measuring the level of a second marker indicative of heart failure.

22. A kit of claim 21, wherein the second marker is a natriuretic peptide.

23. A kit of claim 22, wherein the natriuretic peptide is brain natriuretic peptide (BNP) or N-terminal pro-brain natriuretic peptide (N-BNP).

24. A kit of claim 21, wherein the second marker is C-reactive protein (CRP).

25. A kit of claim 21, wherein the one or more reagents for measuring the second marker comprises an antibody that binds specifically to the second marker.

26. A kit of claim 25, wherein the antibody is a monoclonal antibody.

27. A kit of claim 21, wherein the second marker is urotensin.

28. A method of monitoring the health of a mammalian subject comprising measuring the level of a first marker in a sample of bodily fluid from said mammalian subject, wherein said first marker is myeloperoxidase (MPO).

29. The method of claim 28, wherein the bodily fluid is plasma.

30. The method of claim 28, wherein the level of the first marker is compared with a level of the first marker which is indicative of the absence of heart failure.

31. The method of claim 28, wherein the level of the first marker is compared with a level of the first marker which is indicative of worsening of heart failure in a mammalian subject previously determined to be experiencing heart failure.

32. The method of claim 28, wherein the level of the first marker is compared with a level of the first marker which is indicative of sudden unexpected cardiac death in a mammalian subject previously determined to be experiencing heart failure.

33. The method of claim 28, wherein the level of the first marker is compared with a level of the first marker which is indicative of a response to therapy in a mammalian subject previously determined to be experiencing heart failure.

34. The method of claim 30, wherein the level of the first marker which is indicative of the absence of heart failure is the level of the first marker from one or more mammalian subjects free from heart failure, or a previously determined reference range for the first marker in mammalian subjects free from heart failure.

35. The method of claim 28, wherein the level of the first marker is measured by contacting the sample with an antibody that binds specifically to the first marker and measuring any binding that has occurred between the antibody and at least one species in the sample.

36. The method of claim 35, wherein the antibody is a monoclonal antibody.

37. The method of claim 28, further comprising measuring the level of a second marker indicative of heart failure.

38. The method of claim 37, wherein the second marker is a natriuretic peptide.

39. The method of claim 38, wherein the natriuretic peptide is brain natriuretic peptide (BNP) or N-terminal pro-brain natriuretic peptide (N-BNP).

40. The method of claim 37, wherein the second marker is C-reactive protein (CRP).

41. The method of claim 37, wherein the second marker is urotensin.

42. The method of claim 37, wherein the level of the second marker is compared with a level of the second marker which is indicative of the absence of heart failure.

43. The method of claim 42, wherein the level of the second marker which is indicative of the absence of heart failure is the level of the second marker from one or more mammalian subjects free from heart failure, or a previously determined reference range for the second marker in mammalian subjects free from heart failure.

44. The method of claim 37, wherein the level of the second marker is measured by contacting the sample with an antibody that binds specifically to the second marker and measuring any binding that has occurred between the antibody and at least one species in the sample.

45. The method of claim 44, wherein the antibody is a monoclonal antibody.
